(19) United States
(12) Patent Application Publication
(10) Pub. No.: US 2006/0225746 A1
(43) Pub. Date: Oct. 12, 2006

(54) Method to decrease excessive portal venous inflow during live donor liver transplantation

(75) Inventor: Nicolas Jabbour, Pasadena, CA (US)

Correspondence Address: MCDERMOTT, WILL & EMERY, 4370 LA JOLLA VILLAGE DRIVE, SUITE 700, SAN DIEGO, CA, 92122, US

(73) Assignee: Paik Medicine International

(21) Appl. No.: 11/265,847

(22) Filed: Nov. 2, 2005

Related U.S. Application Data
(60) Provisional application No. 60/624,845, filed on Nov. 3, 2004.

(51) Int. Cl. A61B 19/00

(52) U.S. Cl. 128/898

(57) ABSTRACT

A method of decreasing excessive portal venous inflow during liver transplantation is provided. The technique involves the use of a temporary left portahepatic shunt. This allows portasystemic decompression during implantation of donor liver and diverts mesenteric blood from the donor liver during the crucial period after unclamping in liver transplantation. The method can be used for any type of liver transplantation and is particularly advantageous for live donor liver transplantation.

### FIELD OF THE INVENTION

This application claims the benefit of priority of U.S. Provisional application Ser. No. 60/624,845, filed Nov. 3, 2004, the entire contents of which is incorporated herein by reference.

A method for decreasing excessive portal venous inflow during live donor liver transplantation is provided.

### BACKGROUND OF THE INVENTION

Living donor liver transplantation accounts for approximately 10% of all liver transplants being performed. One problem unique to lobar transplantation relates to hepatic congestion and associated functional impairment of the transplanted lobe. This congestion is multifactorial and includes low graft-weight ratio, middle hepatic vein drainage problems, and excessive portal venous inflow.

Animal data from asplanchnic and anhepatic models implicate the intestine as the source of the tumor necrosis factor that is released into the circulation following portal unclamping. Tumor necrosis factor, in turn, leads to adhesion molecule upregulation and endothelial cell disruption and potentiates allograft dysfunction. Although veno-venous bypass may alleviate some of these problems, it does not prevent excessive mesenteric venous flows, nor does it prevent the tumor necrosis factor production associated with portal clamping during the portal anastomotic phase.

### SUMMARY OF THE INVENTION

A method of decreasing excessive portal venous inflow during liver transplantation is provided. The technique involves the use of a temporary left portahepatic shunt. This allows portasystemic decompression during implantation of donor liver and diverts mesenteric blood from the donor liver during the crucial period after unclamping in liver transplantation.

One embodiment is a method of decreasing venous inflow during a liver transplantation procedure in a patient, by construction of a shunt by an end to end anastomosis of the left portal vein to the left hepatic vein. In one aspect the shunt is produced from the left portal vein to the left hepatic vein. The shunt may be temporary or permanent. In one aspect, the left portal vein is dissected up to the remnant of the umbilical cord prior to creation of the shunt. In a further aspect, a graft is produced to shunt the left portal vein to the left hepatic vein under conditions when an adequate length of portal vein is not available. The graft may be a polytetrafluoroethylene (PTFE) graft. In one aspect, the liver transplantation procedure is selected from the group consisting of: donor liver transplantation, split liver transplantation, and whole cadaveric liver transplant.

A further embodiment is a method of live donor liver transplantation, by: dissecting the portal vein up to the remnant of the umbilical cord; constructing a shunt by an end to end anastomosis of the left portal vein to the left hepatic vein; implanting a donor liver. In one aspect, the shunt is left open until the end of the operation. In a further aspect, the portal vein is doubly ligated prior to closure. In one aspect, the dissection includes ligating collaterals coming off the left portal vein. In a further aspect the collaterals include branches to segments I and IV. In a further aspect, the shunt is left open following liver implantation and is occluded later after the surgery using radiological techniques.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a completed left portahepatic shunt and the right branch of the portal vein.

FIG. 2 shows a completed left portahepatic shunt using PTFE graft, right hepatic vein, left portal vein, left hepatic vein, hepatic artery pedicle, the right portal vein is not visualized in this picture because is it rotated behind.

FIG. 3 shows a completed right portal vein anastomosis, left portahepatic shunt, which can be used as a vent and ligated at the time of closure.

### DETAILED DESCRIPTION OF THE PREFERRED EMBODIMENT

A method using a temporary left protahepatic shunt has been developed which allows portasystemic decompression during implantation and diverts mesenteric blood from the liver during the crucial period after unclamping. Early lobar congestion can be avoided and the shunt may be left in place for as long as 2 hours while volume homeostasis is restored. The technique allows the tailoring of the portal flow to the graft size to avoid any liver congestion or dysfunction. Thus, a method of decreasing excessive portal venous inflow during liver transplantation is provided. The method can be used for any liver transplantation, however it is particularly useful for live donor liver transplantation. The technique involves creating a temporary left portahepatic shunt. This allows portasystemic decompression during implantation of the donor liver and diverts mesenteric blood from the donor liver during the crucial period after unclamping.

Using the method, early lobar congestion can be avoided, and the shunt may be left in place while volume homeostasis is restored. Preferably the shunt is left in place for up to two hours. However, in some circumstances it may be left longer. This technique allows the portal flow to be tailored to the graft size to avoid any liver congestion or dysfunction.

The Technique

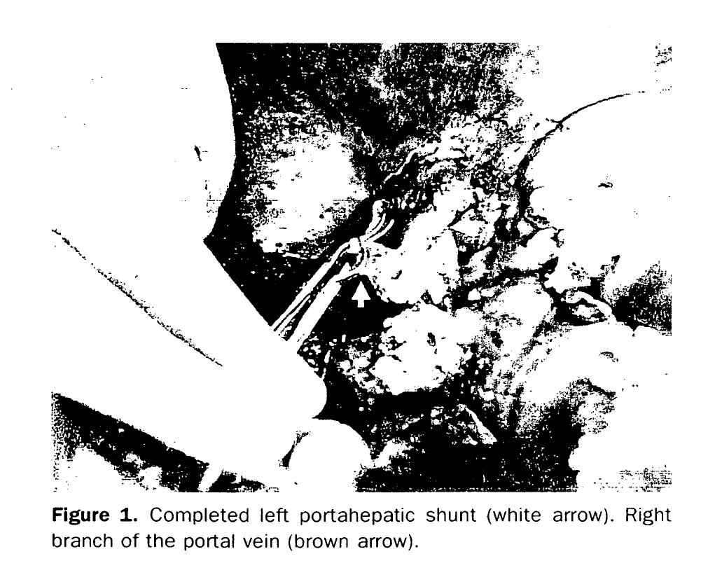

The technique involves the intrahepatic dissection of the left portal vein up to the remnant of the umbilical vein. This is done just prior to liver explantation. This dissection is achieved by ligating a few collaterals coming off the left portal vein. These include segmental branches to the segments I and IV. A shunt is then constructed preferably by doing an end-to-end anastomosis of the left branch of the portal vein to the left hepatic vein (FIG. 1) in a continuous fashion. Preferably this is done using 6-0 Prolene (Ethicon). In FIG. 1, the left portahepatic shunt is shown with a white arrow and the right branch of the portal vein a brown arrow. The procedure usually takes less than 15 minutes.

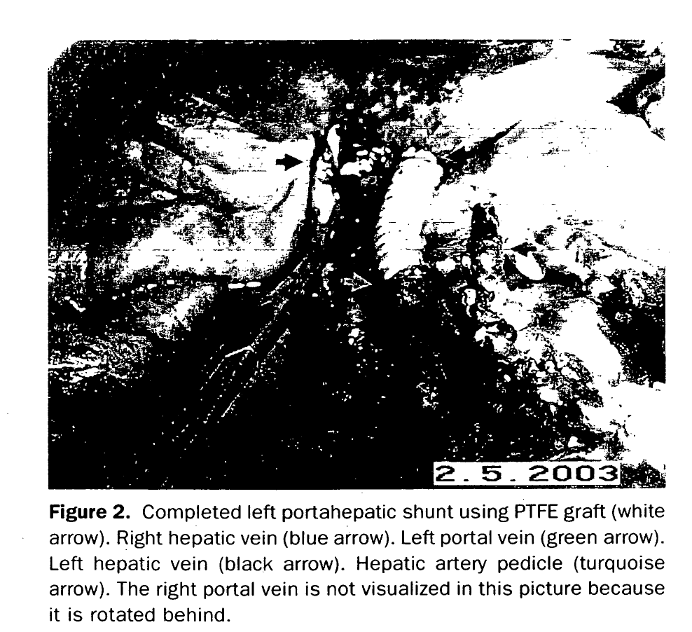

For tumor patients or where an adequate length of portal vein is not available, a PTFE graft was used to create the shunt (FIG. 2). FIG. 2 shows a completed left portahepatic shunt using graft, including but not limited to a PTFE (polytetrafluoroethylene) graft (white arrow), right hepatic vein (blue arrow), left portal vein (green arrow), left hepatic vein (black arrow), hepatic artery pedicle (turquoise arrow), the right portal vein is not visualized in this picture because is it rotated behind.

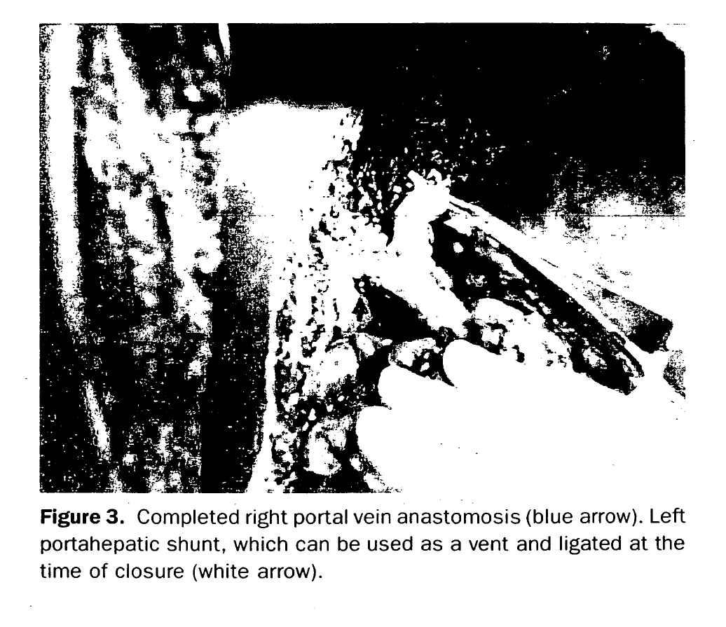

The shunt is left open and is doubly ligated at any time prior to closure (FIG. 3). Where a PTFE graft is used, it is excised and the stump of the hepatic vein is suture-ligated. FIG. 3 shows a completed right portal vein anastomosis (blue arrow), left portahepatic shunt, which can be used as a vent to decrease liver congestion following liver reperfusion in patients with severe portal hypertension or where the implanted liver is too small relative to the recipient body weight. This shunt can be left open for a variable period of time after liver reperfusion, then ligated and excised once it is clear that liver congestion is unlikely (usually 1-2 hour post perfusion). In selected patients this shunt may be left open and eventually closed several days after the transplant procedure. The closure can be effected using interventional radiology once it is deemed appropriate based on portal flow assessment.

The temporary portahepatic shunt allows the portal circulation to be completely decompressed prior to donor liver implantation. It maintains hemodynamic stability throughout the anhepatic phase, reduces reperfusion injury, and decreases graft congestion following implantation of the donor liver. It results in a more successful liver transplantation. In addition, for live donor transplantation, this technique can be used in patients with a small graft size as compared to the size of the recipient or in patients with more advanced liver disease where the graft size requirement may be relatively higher, and provides a more successful result in these patients also.

Application of the disclosed methods is not limited to live donor liver transplantation. Applications of this invention include, but are not limited to, split liver transplantation, and liver transplantation using an entire liver with a relatively small size graft in relation to the recipient weight (for example, a total graft weight of less then 1% of total body weight of the recipient).

This procedure has been performed in over twenty patients undergoing live donor liver transplant (see the Examples below). This group of patients demonstrated the feasibility of the shunt for patients with severe portal hypertension undergoing live donor liver transplantation.

### EXAMPLES

### Example 1

### Use of the Method for Live Donor Liver Transplantation

The intrahepatic dissection of the left portal vein up to the remnant of the umbilical vein, just prior to total hepatectomy of patient's liver, was achieved by ligating a few collaterals coming off the left portal vein. These included segmental branches to the segments I and IV. A shunt was then constructed by doing an end-to-end anastomosis of the left branch of the portal vein to the left hepatic vein in a continuous fashion. This was done using 6-0 Prolene (Ethicon). The procedure took less than 15 minutes. The shunt was left open and was doubly ligated prior to closure.

The method was performed on 20 patients as a right lobe liver transplant, these patients have relatively higher portal pressure than other patients and all of the grafts functioned properly. The shunt was performed prior to liver explantation, and was closed between one to two hours following implantation. Preliminary results suggest that the method offers a number of advantages with respect to patient and graft survival.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. A method of decreasing venous inflow during a liver transplantation procedure in a patient, comprising:
construction of a shunt by an end to end anastomosis of the left portal vein to the left hepatic vein.

2. The method of claim 1 where said shunt is produced from the left portal vein to the left hepatic vein.

3. The method of claim 1 wherein said shunt is temporary.

4. The method of claim 2, wherein the left portal vein is dissected up to the remnant of the umbilical cord prior to creation of the shunt.

5. The method of claim 1, further comprising producing a graft to shunt the left portal vein to the left hepatic vein under conditions when an adequate length of portal vein is not available.

6. The method of claim 1, wherein the graft is a PTFE graft.

7. The method of claim 1, wherein the liver transplantation procedure is selected from the group consisting of: donor liver transplantation, split liver transplantation, and whole cadaveric liver transplant.

8. A method of live donor liver transplantation, comprising:
dissecting the portal vein up to the remnant of the umbilical cord;
constructing a shunt by an end to end anastomosis of the left portal vein to the left hepatic vein;
implanting a donor liver.

9. The method of claim 8 wherein said shunt is left open until the end of the operation.

10. The method of claim 8 wherein said dissection comprises ligating collaterals coming off the left portal vein.

11. The method of claim 10 wherein said collaterals include branches to segments I and IV.

12. The method of claim 10, wherein the shunt is left open following liver implantation and is occluded later after the surgery using radiological techniques.
